(19) United States
(12) Patent Application Publication
(10) Pub. No.: US 2012/0022620 A1
(43) Pub. Date: Jan. 26, 2012

(54) WARMING THERAPY DEVICE INCLUDING HEATED MATTRESS ASSEMBLY

(75) Inventors: Andrei Khodak, Hatfield, PA (US); Frank Mett, Luebeck (DE); Peter D. Sabota, Warminster, PA (US); Kanti Patel, King of Prussia, PA (US); Mark A. Phillips, King of Prussia, PA (US)

(73) Assignee: DRAEGER MEDICAL SYSTEMS, INC., Telford, PA (US)

(21) Appl. No.: 13/146,596

(22) PCT Filed: Mar. 16, 2010
(86) PCT No.: PCT/US2010/027379

§ 371 Date: Jul. 27, 2011

Related U.S. Application Data
(60) Provisional application No. 61/161,184, filed on Mar. 18, 2009.
(60) Provisional application No. 61/170,173, filed on Apr. 17, 2009.

(51) Int. Cl. A61F 7/08; H05B 3/02; A61B 6/04; A61F 7/00; A47C 21/04

(52) U.S. Cl. 607/96; 5/421; 5/601; 219/546

(57) ABSTRACT

An apparatus and method for performing warming therapy is described. In one exemplary embodiment, the apparatus includes a patient support assembly and at least one heating element coupled to the patient support assembly, formed of a plurality of heating cells. In another exemplary embodiment, the heating element is formed of a plurality of separate heating segments which may be Individually controlled. In yet another exemplary embodiment, the heating element is coupled to at least one oxygen sensor and a control system for controlling the power applied to the heating element based on the oxygen level measured by the oxygen sensor. The heating element may be made of materials which are X-ray transparent, so that a patient disposed on a heated mattress will not need to be moved from the mattress to have effective X-ray diagnostics performed.

### CROSS REFERENCE TO RELATED APPLICATIONS

This application claims priority to U.S. Provisional Application Ser. No. 61/161,184, filed Mar. 18, 2009, and U.S. Provisional Application Ser. No. 61/170,173, filed Apr. 17, 2009, the entire contents of both of which are hereby incorporated by reference, as if fully set forth herein.

### FIELD OF THE INVENTION

This present invention relates generally to a method and apparatus for performing warming therapy on medical patients. More particularly, the present invention relates to a method and apparatus for heating a mattress in a warming therapy device, wherein the heating apparatus is X-ray transparent, and wherein the heating system includes an oxygen monitoring system for providing control feedback to control the power applied to the heating apparatus.

### BACKGROUND OF THE INVENTION

Conventional warming therapy devices achieve patient warming through the use of air pumps and convective and radiant heaters. Some existing devices also use a conductive method of warming therapy, or a heated mattress. One such device (which is manufactured by the present assignee and sold under the name Babytherm®) utilizes an electric resistive heater attached to a metal plate which supports a thermally conductive gel mattress. The metal plate design provides some degree of uniform temperature distribution across the surface of the mattress, however, it is also non-transparent to X-rays. So, an X-ray cassette can only be placed over the mattress support containing the metal plate, which may interfere with the care of the patient disposed on the mattress. Other conventional warming devices including conductive warming utilize X-ray transparent carbon fiber material instead of a metal plate.

For example, U.S. Pat. Nos. 6,814,889 and 6,974,935, the disclosures of which are hereby incorporated by reference into this application, discuss a heated mattress device 1 which includes a conductive layer 3 with carbon particles. Carbon fiber is electrically conductive, and thus allows a similar distribution of heat as provided by the metal plate, and also permits the placement of an X-ray cassette below the carbon fiber layer. However, such structures often leaves artifacts on the X-ray image due to non-X-ray transparent temperature control sensors, buses and connecting wires disposed in the area around the carbon fiber layer.

The above-described metal plate design is also not usable in ‘oxygen rich’ environments because it consumes more power than is acceptable under current International Electrotechnical Commission (EC) guidelines. Oxygen rich environments could include, for example, environments where the oxygen level was four percent (4%) or more above ambient, as per IEC guidelines. For example, current IEC guidelines place an upper limit of 15 Volt-Amperes (VA) per circuit in oxygen rich environments, and the above-described metal plate design exceeds those limits.

Existing carbon fiber beaters are similarly unusable in ‘oxygen rich’ environments because of the above-described IEC guidelines. Although the above-referenced patents described temperature sensors for controlling heating, such sensors do not operate to maintain power consumption levels below IEC guidelines for oxygen rich environments. Rather, they assist only in achieving and maintaining a desired temperature for the mattress, with regard to the surrounding environment.

The present invention overcomes the deficiencies of conventional warming therapy devices through the use of a heated mattress assembly. In particular, conventional warming therapy devices are unable to maintain a desired temperature distribution at the interface between the mattress and the patient. The present invention solves this problem by using temperature sensors to measure the temperature at the interface between mattress and the patient, and using the temperature measurements to control specific heating elements in order to achieve a desired temperature at the interface between mattress and baby. In addition, the present invention avoids the above-described artifact problem experienced by warming therapy devices including carbon fiber structures within the mattress support by utilizing temperature sensors, buses and supply wires comprised of non-metallic radiolucent conductive materials, such as carbon nanotubes.

Further, in one exemplary embodiment, the present invention utilizes a segmented heated mattress assembly including an Oxygen control Device (OCD). In particular, conventional warming therapy devices are unable to achieve and maintain different temperatures depending on the presence or absence of oxygen in the surrounding environment. The present invention solves this problem by using the OCD to control the power supplied to one or more heating segments of a mattress, so as to achieve a desired temperature at the interface between mattress and baby and to also avoid power levels which exceed IEC guidelines.

Accordingly, there is presently a need for a warming therapy device that permits efficient heating of patients in oxygen rich environments, and simultaneously allows for simple and effective X-ray examinations to be performed in the same environment.

### SUMMARY OF THE INVENTION

An exemplary embodiment of the present invention comprises an apparatus including a patient support assembly and at least one heating element coupled to the patient support assembly, the at least one heating element formed of a plurality of heating cells.

An exemplary embodiment of the present invention also comprises an apparatus including a patient support assembly and at least one heating element coupled to the patient support assembly, the at least one heating element formed of an insulating layer and first and second support layers, such that the insulating layer is disposed between the first and second support layers.

An exemplary embodiment of the present invention also comprises a heating element including a first support layer, a heater layer coupled to the first support layer, an insulating layer coupled to the heater layer, a grid layer coupled to the insulating layer, and a second support layer coupled to the grid layer.

An exemplary embodiment of the present invention also comprises an apparatus including a patient support assembly and at least one heating element coupled to the patient support assembly, the at least one heating element formed of a plurality of heating segments.

An exemplary embodiment of the present invention also comprises an apparatus including a patient support assembly and at least one heating element coupled to the patient support assembly, the at least one heating element including power rails disposed on opposing sides thereof, and at least one temperature sensor coupled thereto.

An exemplary embodiment of the present invention also comprises a control system including at least one heating element, a system controller coupled to the at least one heating element, and at least one oxygen sensor coupled to the system controller, wherein the at least one oxygen sensor and the system controller operate to control the power applied to the at least one heating element.

An exemplary embodiment of the present invention also comprises a method of providing warming therapy to a patient, the method including providing at least one heating element in proximity to a patient, and selectively activating the at least one heating element, so that the patient is heated to a predetermined level.

### BRIEF DESCRIPTION OF THE DRAWINGS

FIG. 1 is perspective view of a warming therapy device according to a first exemplary embodiment of the present invention.

FIG. 2 is an overhead perspective view of the warming therapy device of FIG. 1.

FIG. 3 is a simplified side cross-section view of the warming therapy device of FIG. 1.

FIG. 4 is a top view with detail of a heating element according to first exemplary embodiment of the present invention.

FIG. 5 is a simplified side cross-section view of a warming therapy device according to a second exemplary embodiment of the present invention.

FIG. 6 is a top view of a heating element according to a second exemplary embodiment of the present invention.

FIG. 7 is a side view of the heating element of FIG. 6.

FIG. 8 is a detail side view of the heating element of FIG. 6, shown along Detail A in FIG. 7.

FIG. 9 is a top cross-section view of the heating element of FIG. 6 which shows the thermocouple grid and an associated controller.

FIG. 10 is a top cross-section view of a heating element according to a third exemplary embodiment of the present invention.

FIG. 11 is a top cross-section view of a heating element according to a fourth exemplary embodiment of the present invention.

FIG. 12 is a top cross-section view of a heating element according to a fifth exemplary embodiment of the present invention.

FIG. 13 is a block diagram of an oxygen control system according, to a first exemplary embodiment of the present invention.

### DETAILED DESCRIPTION

The present invention relates to a warming therapy device (e.g., incubator, warmer, etc.) including a heated mattress tray assembly. In particular, the warming therapy device includes a resistive or radiolucent heater disposed within a mattress tray assembly for providing heating of a patient disposed on the mattress tray assembly in one embodiment. In another embodiment, the warming therapy device includes a segmented carbon fiber heater coupled to an oxygen control device and disposed within a mattress tray assembly for providing heating of a patient situated on the mattress tray assembly.

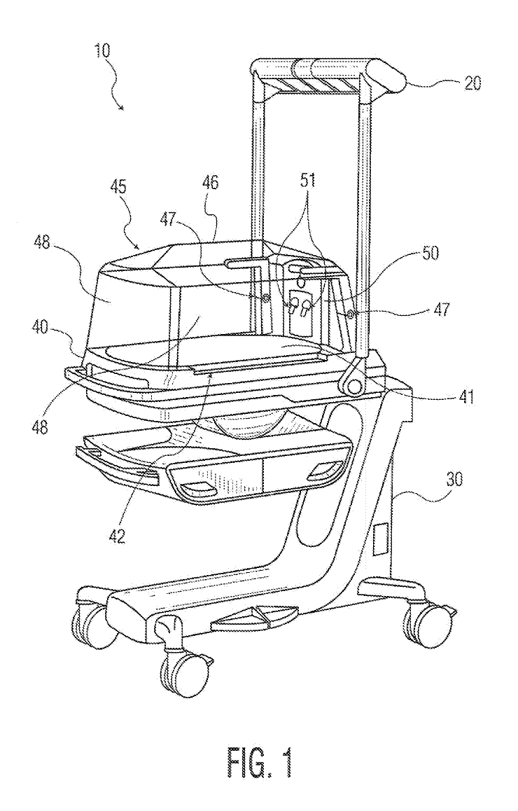

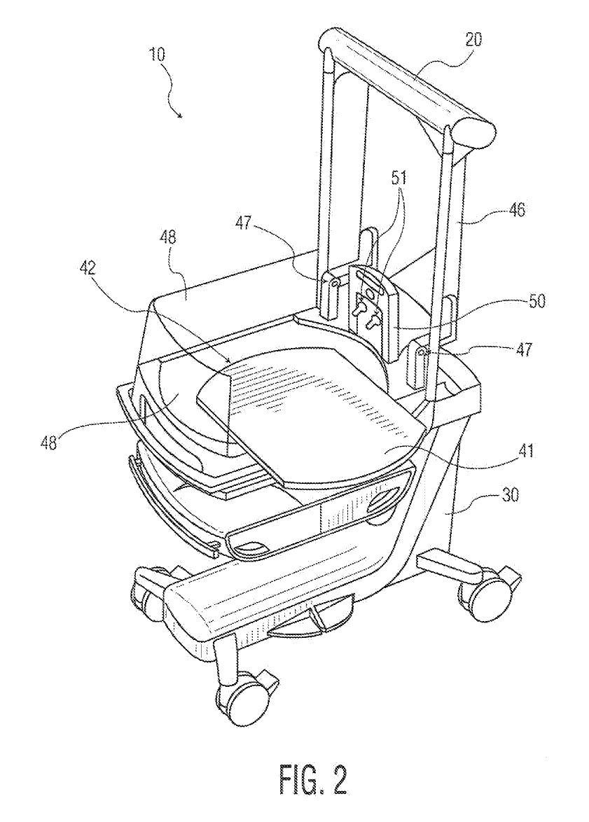

FIGS. 1 and 2 show a warming therapy device 10 according to a first exemplary embodiment of the present invention. The warming therapy device 10 includes a radiant heater head 20, and a patient support assembly 30 including a mattress tray assembly 40. The mattress tray assembly 40 may include a hood 45 which has a top portion 44 which pivots about one or more axes 47. The hood 45 may also include one or more sidewalk 48 which may be slideable, removable, pivotable or rotatable. The mattress tray assembly 40 includes a mattress tray 41 disposed within a recessed support base 42.

The mattress tray 41 may be made rotatable within the support base 42 up to three hundred and sixty degrees (360°). FIG. 2 specifically shows the mattress tray 41 rotated approximately ninety degrees (90°) with respect to the position shown in FIG. 1. FIG. 2 also shows the top portion 46 of the hood 45 rotated up so that it is approximately ninety degrees (90°) with respect to the mattress tray 41, in the exemplary embodiment shown in FIG. 2, the side walls 48 of the hood 45 are capable of sliding vertically within a portion of the mattress tray assembly 40, so that they may become disposed, partially or completely, below the plane of the mattress tray 41.

The warming therapy device 10 may optionally include a backplane 50, to which ventilation hoses and other devices may be coupled through, for example, interconnection nozzles 51. The backplane 50 may also include one or more oxygen sensors disposed therein for sensing an oxygen level inside the hood 45, as explained in detail below.

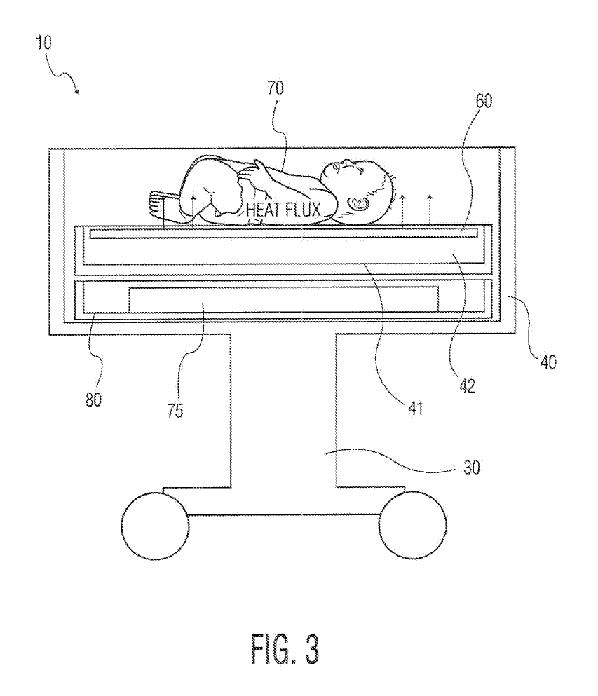

FIG. 3 shows a simplified side cross-sectional view of the warming therapy device 10 shown hi FIGS. 1 and 2. The mattress tray assembly 40, including mattress tray 41 and mattress 42, are shown disposed on the patient support assembly 30. Disposed within the mattress 42 is an electric resistive heating element 60 for providing heat to a patient 70 disposed on the mattress. As discussed in detail below, the heating element 60 may be formed of a single piece, or may be divided into a plurality of separate portions, or segments. A utility tray 80 may be disposed beneath the mattress tray 41, and may house an X-ray cassette 75. The X-ray cassette 75 may be used, for example, in performing X-ray examinations of the patient 70, as is well known in the art.

The resistive heating element 60 may be formed of a substantially flat member or plurality of substantially flat members (segments) which extend through some or all of the mattress 42. For example, if the mattress 42 is substantially rectangular in shape as shown in FIGS. 1 and 2, the resistive heating element 60 may be formed as a rectangle with similar dimensions, or a plurality of rectangular segments. The resistive heating element 60 may be formed of materials with similar structural parameters as the materials for the mattress 42, such as (1) thin metal cloth or foil (e.g., Aluminum foil), (2) non-metallic conductive film (e.g., graphite or graphene film), or (3) cloth made from non-metallic conductive yarn (e.g., carbon fiber or carbon nanotube material), insuring the overall softness and flexibility of the mattress are maintained. Carbon nanotube material is available from various commercial sources, including from CNT Technologies (Seattle, Wash.) and Nanocomp Technologies (Concord, N.H.). The resistive beating element 60 may be placed within the mattress 42 so as to efficiently transmit heat to the patient 70, but also so as to prevent excessive heating of the members below the mattress. For example, the resistive heating element 60 may be placed in the mattress 42 at a position closer to the patient 70, so that the material of the mattress acts as an insulator with respect to the X-ray cassette 75, and any other devices located beneath the mattress 42.

As mentioned above, non-metallic conductive materials may be used for the resistive heating element 60. For example, materials using carbon nanotubes may be utilized to achieve an increase in electrical and thermal conductivity. Materials such as graphene may also be used for the resistive heating element 60. Graphene in particular shows electrical conductivity even as one (1) atom layer thick film, and is virtually undetectable by microscope. Conductive plastics, such as optically transparent vacuum metallized polyethylene, may also be used for the resistive heating element 60. However, in order to insure effective X-ray imaging of the patient 70, any material used for the resistive heating element 60 should be X-ray transparent, which is the case for some conductive plastics and thin metal cloth. Those of ordinary skill will realize that the X-ray cassette 75 is not a necessary element of the above-described exemplary embodiment, and in cases where X-ray capacity is not needed the resistive heating element 60 may be formed of materials which are not necessarily X-ray transparent.

The thermal capacity of the mattress 42 may be made sufficiently large so as to allow continued warming of the patient 70 even after the electric heating element 60 is turned off. Certain gels, such as polyurethane and silicone gels, may be used for the forming the entirety, or a portion of die mattress 142 as they have a high thermal capacity. The previously-mentioned Babytherm® product manufactured by the present assignee includes such a gel within the mattress. However, those of ordinary skill in the art will realize that other materials (e.g., thermally conductive plastic foam) are also suitable for forming the mattress 42, or some portion thereof.

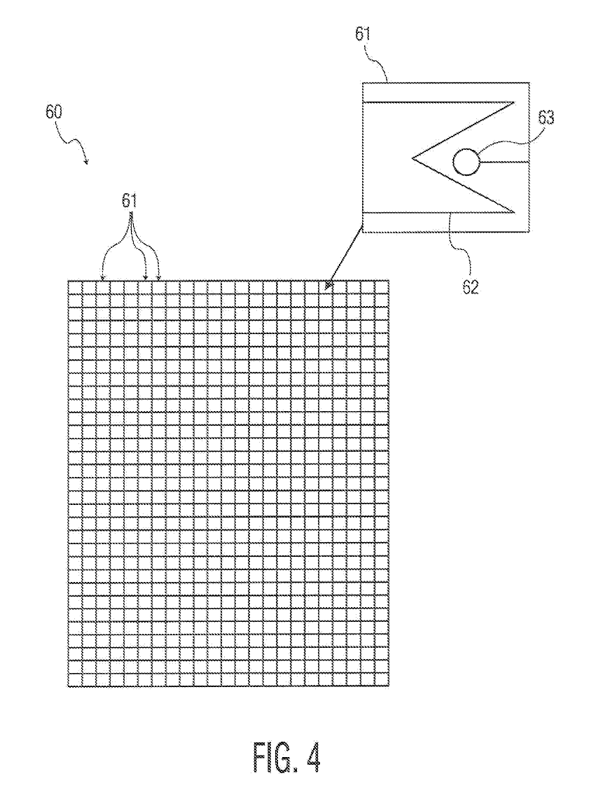

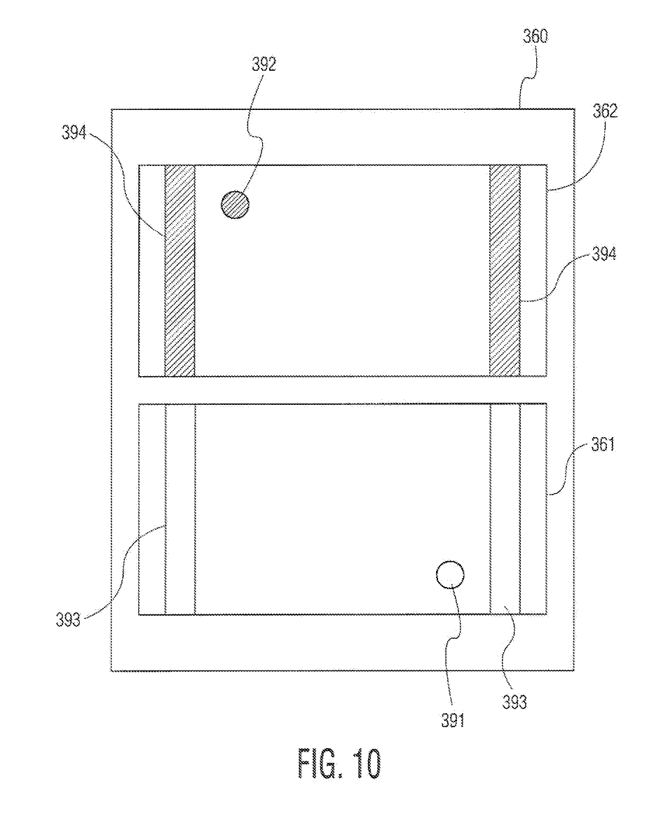

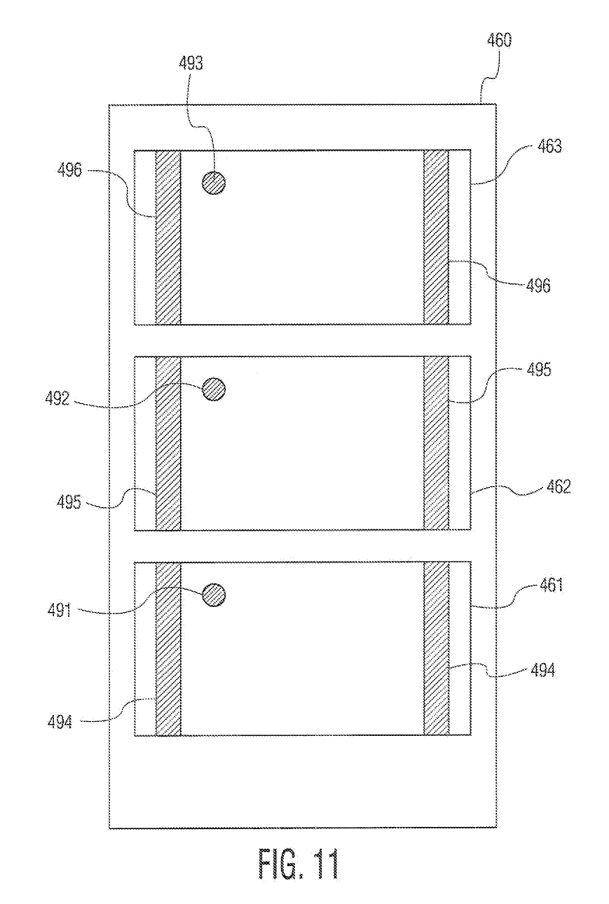

FIG. 4 shows details of a heating element 60 according to first exemplary embodiment of the present invention. As shown, the resistive heating element 60, or each segment thereof (when using a segmented heating element such as shown and described in connection with FIGS. 10-12 below), may be formed as a matrix structure, so as to provide for substantially uniform temperature distribution across the heating element or each separate segment. Such a structure provides substantially uniform temperature distribution across the surface of the heating element. In particular, the resistive heating element 60 may be divided into a plurality of cells 61, each containing a heating element 62 controlled by a sensor 63. This type of configuration allows each cell 61 to be powered on or of depending upon the temperature measured by the sensor 63 associated with that cell. As noted above, X-ray transparent materials, such as conductive plastics and thin metal cloth, may be selected for forming the heating elements 62 and sensors 63 in order to permit effective X-ray imaging of the patient 70. As an alternative to the cells 61 including heating elements 62 and sensors 63 discussed above, Positive Temperature Coefficient (PTC) heaters may be used in a matrix arrangement for the resistive heating element 60. PTC heaters are typically formed from a special compound which may increase the thermal resistance of the heater at a particular temperature level. This characteristic provides a self-controlling mechanism whereby the heater temperature can be stabilized at that particular level. Additionally, materials with a strong dependence of electrical conductivity on temperature (such as carbon nanotube materials) can be used in a matrix structure simultaneously, as heaters and temperature sensors.

FIG. 5 shows a simplified side cross-sectional view of a warming therapy device 110 according to a second exemplary embodiment of the present invention. The warming therapy device 110 is similar to the warming therapy device 10 discussed above, and like reference numerals denote like elements. The warming therapy device 110 may include an electric resistive heating element 160, which is similar in structure to the resistive heating element 60 shown in FIG. 4.

The warming therapy device 110 includes a mattress tray assembly 140, including a mattress tray 141 and mattress 142, which are disposed on a patient support assembly 130. As opposed to the warming therapy device 10, an electric resistive heating element 160 is disposed on the mattress tray 141, for providing heat to a patient 170 disposed on the mattress 142. A utility tray 180 may be disposed beneath the mattress tray 141, and may house an X-ray cassette 175. The X-ray cassette 175 may be used, for example, in performing X-ray examinations of the patient 170, as is well known in the art.

The resistive heating element 160 may be a substantially flat member which extends through some or all of the mattress tray 141. For example, if the mattress tray 141 is substantially rectangular in shape as shown in FIGS. 1 and 2, the resistive heating element 160 may be formed as a rectangle with similar dimensions. As noted above, the resistive heating element 160 may be formed of materials such as: (1) thin metal cloth or foil (e.g., Aluminum foil), (2) non-metallic conductive film (e.g., graphite or graphene film), or (3) cloth made from non-metallic conductive yarn (e.g., carbon fiber or carbon nanotube material). In the second exemplary embodiment, the material for the mattress 142 may be selected to have high thermal conductivity, so as to effectively transmit heat from the resistive heating element to the patient 170. For example, the mattress 142 may be formed of partially or completely of thermally conductive gel or plastic foam.

As with the resistive heating element 60 discussed above, non-metallic conductive materials may be used for the resistive heating element 160. For example, materials using carbon nanotubes may be utilized to achieve an increase in electrical and thermal conductivity. Materials such as graphene may also be used for the resistive heating element 160. Graphene in particular shows electrical conductivity even as one (1) atom layer thick film, and is virtually undetectable by microscope. Conductive plastics, such as optically transparent vacuum metallized polyethylene, may also be used for the resistive heating element 160. However, in order to insure effective X-ray imaging of the patient 170, any material used for the resistive heating element 160 should be X-ray transparent, which is the case for some conductive plastics and thin metal cloth. Those of ordinary skill will realize that the X-ray cassette 175 is not a necessary element of the above-described exemplary embodiment, and in cases where X-ray capacity is not needed the resistive heating element 160 may be formed of materials which are not necessarily X-ray transparent.

The thermal capacity of the mattress 142 may be made sufficiently large so as to allow continued warming of the patient 170 even after the electric heating element 160 is turned off. Certain gels, such as polyurethane and silicone gels, may be used for the forming the entirety, or a portion of the mattress 142, as they have a high thermal capacity. The previously-mentioned Babytherm® product manufactured by the present assignee includes such a gel within the mattress. However, those of ordinary skill in the art will realize that other materials (e.g., thermally conductive plastic foam) are also suitable for forming the mattress 142, or some portion thereof.

Again, as with the resistive heating element 60 discussed above, the resistive heating element 160 may be formed as a matrix structure, as shown in FIG. 4. Such a structure provides substantially uniform temperature distribution across the surface of the heating element 160. As also noted above, PTC heaters may be used in a matrix arrangement for the resistive heating element 160.

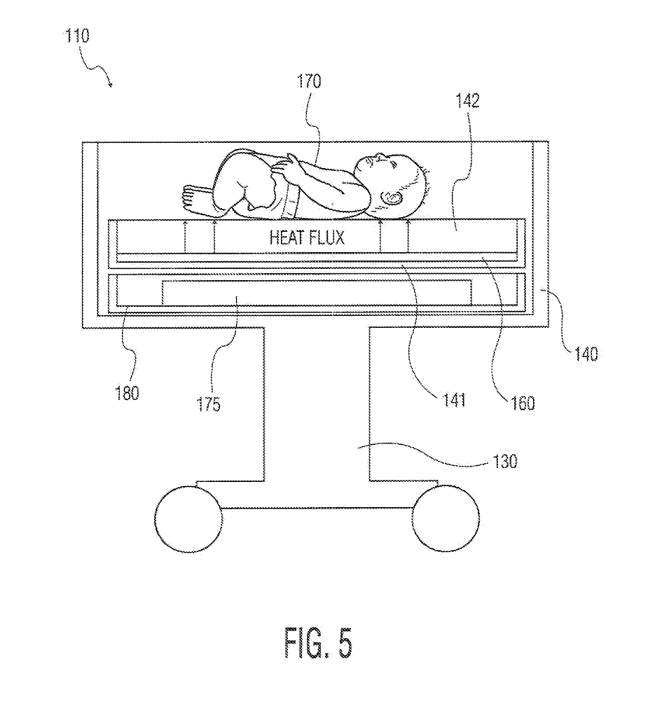

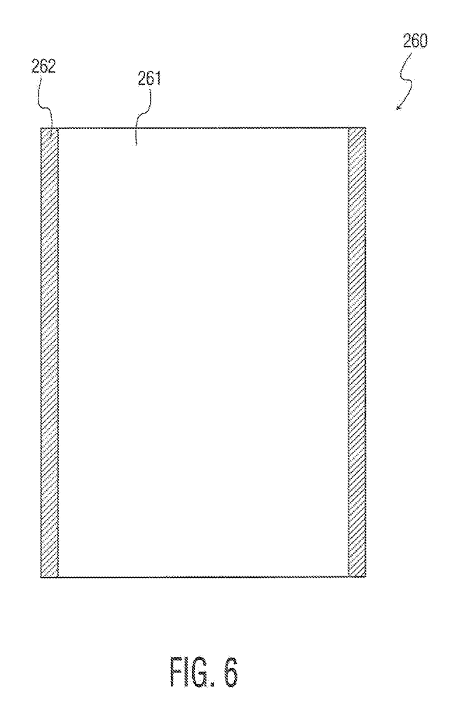

FIG. 6 shows a top view of a heating element 260 according to a second exemplary embodiment of the present invention. This heating element 260 may be used in connection with a mattress tray assembly (including a mattress tray and mattress) and warming therapy device, such as the mattress tray assemblies 40 and 140, and warming therapy devices 10, 110, discussed above with regard to the first and second exemplary embodiments. In particular, the heating element 260 may be placed in close proximity to a patient (such as in a mattress or mattress tray assembly), in order to provide heating of the patient.

When, for example, a patient is disposed on or in close proximity to the heating element 260, the temperature directly below the patient (at the interface) increases due to the fact that heat dissipation to the ambient environment is at least partially blocked by the patient's body. The heating element 260 according to the second exemplary embodiment of the present invention overcomes this increased heating problem through the use of a thermocouple grid (i.e., temperature sensing array), as explained below.

As shown in FIG. 6, the heating element 260 may comprise a radiolucent heating element 261, such as a carbon film heater (made from, for example, carbon-filled polymer film or carbon nanotube film), which is bordered on at least two (2) sides by electrically conductive bus bars 262. The bus bars 262 may be coupled to a source of electrical power, such as a voltage source. The bus bars 262 extend through the entire cross-section of the heating element 261, whereas the radiolucent heating element may be comprised of various layers, as discussed below. The bus bars 262 may be formed of an electrically conductive non-radiolucent materials (such as, for example, Copper mesh, Copper plated film), or radiolucent materials (e.g., carbon fiber), or a blend of both.

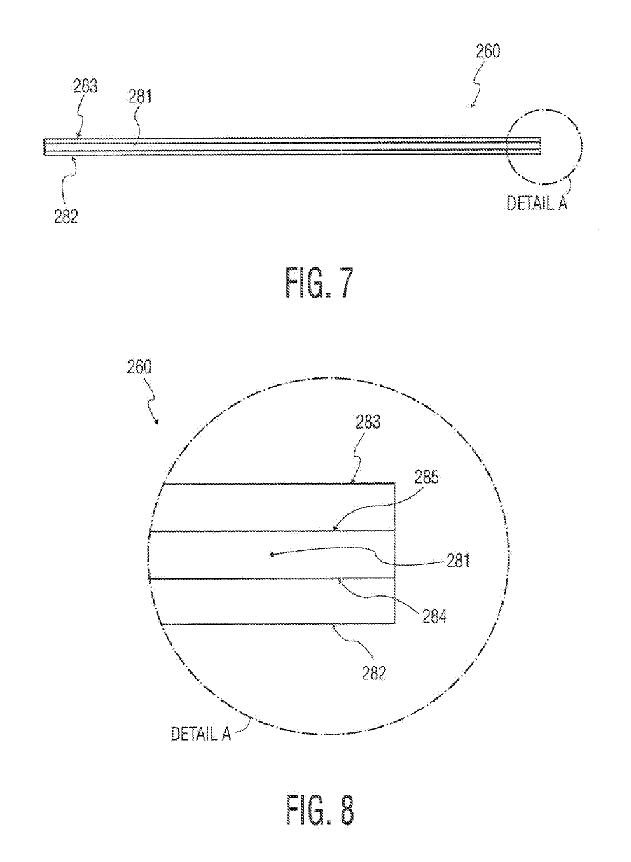

FIG. 7 shows a side view of the heating element 260. As shown, the heating element 260 may be comprised of at least three (3) layers, an insulating layer 281 which is sandwiched by two (2) support layers 282, 283. The insulating layer 281 may be formed of thin, flexible electrically insulating plastic (e.g., polyester, polyurethane) film. The support layer 282 may be formed of foam materials such as polyurethane or polyester foam. The support layer 283 may be formed of a material for improving patient comfort, such as an expanded polyester pad. The thickness of the support layers 282, 283 contributes to the heat conducted to the patient. Ideally, the support layer 282 offers an insulating property to prevent heat loss from the bottom surface of the heating element 284, whereas support layer 283 is more thermally conductive than support layer 282, thus providing patient comfort and more efficient heat transfer to the patient. All the layers of the heating element 260 may be sealed with a waterproof outer covering that may be made out of synthetic or natural fabric materials. In such an embodiment, the patient would lie on top of the waterproof outer covering.

FIG. 8 shows a detail side view of the heating element 260, along Detail A shown in FIG. 7. FIG. 8 shows the insulating layer 281, and support layers 282, 283, as well as a carbon film heater layer 284 (sandwiched between the support layer 282 and the insulating layer 281) and a thermocouple grid layer 285 (sandwiched between the support layer 283 and the insulating layer 281). Thus, the heating element 260 may be formed of at least five (5) layers: a first support layer 282, carbon film beater layer 284, an insulating layer 281, a thermocouple grid layer 285, and a second support layer 283. Both the carbon film heater layer 284 and the thermocouple grid layer 285 may be formed of radiolucent carbon film. One of the benefits of using radiolucent carbon film for the heater layer 284 and the thermocouple grid layer 285 is that it is X-ray transparent, thereby allowing X-ray procedures to be performed on a patient disposed on or near the heating element 260.

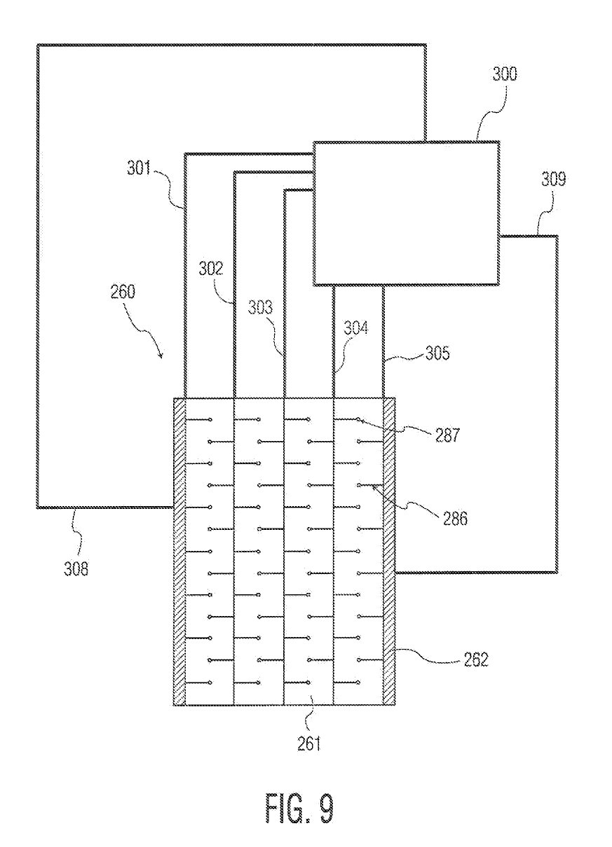

FIG. 9 shows a top cross-sectional view of the heating element 260 which shows the structure of the thermocouple grid layer 285. The thermocouple grid layer 285 may be comprised of a series of electrically conductive leads 286 which terminate in respective thermocouples 287. The thermocouples 287 operate to sense the temperature at various portions of the heating element 260, and may be used to maintain a pre-selected temperature for the heating element. The spacing of the thermocouples 287 along the thermocouple grid layer is preferably less than the contact area of the patient (disposed on the mattress 242) with the heating element 260, so that at least one thermocouple is always covered by the patient. Such an arrangement also permits the temperature at the interface between the heating element 260 and the patient to be maintained at the pre-selected value. The thermocouples 287 may be made from radiolucent carbon nanotube materials. Each thermocouple 287 provides temperature information to a mattress controller 300. The mattress controller 300 regulates the electric power supplied to the heating element 260 via power bus bars 262, based on the temperature information provided by the thermocouples 287 and a predetermined mattress temperature (which may be set by a user or caregiver). The mattress controller compares the predetermined temperature against the data from the thermocouples 287 and controls the power to the heating element 260. The temperature at each bus of thermocouples 287 may be measured on one or more control lines 301-305, and provided to the controller 300 for comparison to the predetermined temperature. Power control lines 308, 309 are coupled to the controller 300 and the power bus bars for providing regulation of the power supplied to the bus bars, and thus the heat of the heating element 260.

Although the mattress tray assemblies 40, 140 according to the first and second exemplary embodiments are shown and described above with reference to an associated warming therapy devices 10, 110 of specific configurations, those of ordinary skill in the art will realize that the mattress tray assemblies 40, 140 may be integrated into any suitable incubator, warmer, medical treatment device or other equivalent apparatus.

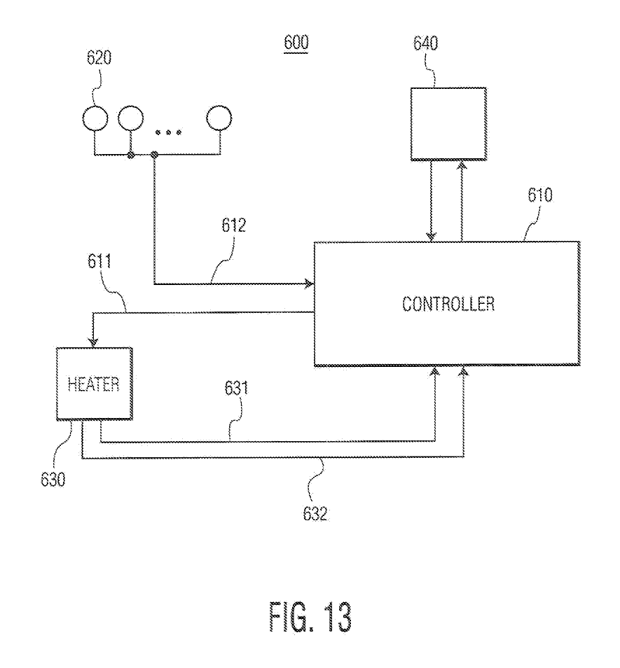

FIG. 10 shows a heating element 360 according to a third exemplary embodiment of the present invention. The heating element 360 may be used in connection with a mattress tray assembly (including a mattress tray and/or mattress) and/or a warming therapy device, such as the mattress tray assemblies 40, 140, and warming therapy devices 10, 100, discussed above with regard to the first and second exemplary embodiments. The heating element 360 may be divided into a plurality of segments in the third exemplary embodiment, including a first segment 361 and a second segment 362. The first segment 361 may be coupled to a first sensor 391 for sensing the temperature of the first segment, and providing a control signal for use in regulating such temperature to a system controller 610 (as shown in FIG. 13). Similarly, the second segment 362 may be coupled to a second sensor 392 for sensing the temperature of the second segment, and providing a control signal to the system controller 610 for use in regulating such temperature. The first and second segments 361, 362 may also include respective power rails or buses 393, 394 for receiving power from an external voltage or current source, which may be part of the system controller 610. As noted above, the first and second segments 361, 362 are preferably radiolucent (e.g. X-ray transparent or translucent) and designed using primarily carbon fiber materials (e.g., carbon films, carbon nanotubes, etc).

Each of the first and second segments 361, 362 of the heating element 360 may be power-limited according specific standards. For example, each of the segments 361, 362 may have an upper power limit of 15 VA, thereby permitting a total power of the heating element 360 to be up to 30 VA. As noted above, current IEC guidelines place an upper limit of 15 VA per circuit, and thus the heating element according to the third exemplary meets IEC guidelines.

The heating element 360 may also include an Oxygen Control Device (OCD) which controls the power supplied to the heating segments 361, 362 between lower and upper limits. For example, a lower power limit may be the IEC guideline for oxygen rich environments of 15 VA, and the upper limit may be greater than 50 VA (for environments which are not oxygen rich). The upper limit may be set so as to insure that the mattress surface temperature does not exceed 42° C. The OCD may additionally control the amount of oxygen supplied to the patient 70 disposed on the mattress 42 (when utilizing the heating element 360 in connection with the warming therapy device 10).

In operation, a user (e.g., health care professional) selects a desired mattress temperature (e.g., 39° C.), and the OCD controls the power supplied to the heating segments 361, 362, and the oxygen supply, so that the mattress (e.g. mattress 42) reaches the desired temperature in the shortest possible time given conditions. For example, if oxygen is not immediately required for the patient during warm up, the OCD would allow maximum power to be supplied to the heating segments 361, 362 (e.g., 70-100 VA) until the desired temperature was reached. Once the desired temperature was reached, the OCD would initiate supply of oxygen to the patient, and would maintain the temperature at the desired level through switching the power to the heating segments 361, 362 on and off at power levels at or below LEC limits (e.g., 15 VA or less). However, if oxygen is required for the patient during warm up, the OCD would regulate the power supplied to the heating segments 361, 362, so as not to exceed 15 VA per segment, which would necessarily increase the warm up time.

FIG. 13 shows as control system 600 for use in connection with the heating element 360 and warming therapy devices 10, 110 according to the first and second exemplary embodiments of the present invention. The control system 600 includes a system controller 610, one or more oxygen sensors 620, a heating element 630 (e.g., heating element 360 described above), and a display and control unit 640. The system controller 610, which may comprise a specially-programmed Central Processing Unit (CPU) or the like, is adapted to supply power to the heating element 630 over first control line 611, and receive one or more control signals from the one or more oxygen sensors 620 over second control line 612. The system controller 610 is also adapted to receive one or more control signals on first and second sensor control lines 631, 632 from the heating element 630. The system controller 610 may also communicate with the display and control unit 640, to display information thereon, and to receive information therefrom. For example, the display and control unit 640 may comprise a touch screen display which is capable of accepting user commands and displaying information.

The first control line 611 may be coupled to the power rails or buses of the heating element 630 (e.g., buses 393, 394 of the heating element 360), to enable the controller 610 to supply power to the heating element. Similarly, the second control line 612 may be coupled to the backplane of a warming therapy device (e.g., backplane 51 of the warming therapy device 10), which may include one or more oxygen sensors (e.g., oxygen sensors 630) as noted above. The first sensor control line 631 may be coupled to a first sensor of the heating element 630 (e.g., sensor 391), and die second sensor control line 632 may be coupled to a second sensor of the heating element (e.g., sensor 392). The system controller 610 may be used to set a predetermined mattress temperature for monitoring purposes. Such a predetermined temperature may be set (and changed) by a health care professional using, for example, the display and control unit 640.

FIG. 11 shows a simplified top cross-section view of a heating element 460 according to a fourth exemplary embodiment of the present invention. The heating element 460 is similar to the heating element 360, according to the third exemplary embodiment in several respects, and in most cases, like reference numerals denote like elements. The heating element 460 may be used in connection with a mattress tray assembly (including a mattress tray and/or mattress) and/or a warming therapy device, such as the mattress tray assemblies 40, 140, and warming therapy devices 10, 100, discussed above with regard to the first and second exemplary embodiments.

The heating element 460 is divided into a plurality of segments in the fourth exemplary embodiment, including a first segment 461, a second segment 462, and a third segment 463. As with the third exemplary embodiment, the first segment 461 may be coupled to a first sensor 491, the second segment 462 may be coupled to a second sensor 492, and the third segment 463 may be coupled to a third sensor 493 all for sensing the respective temperatures of the first through third segments, and providing control signals for use in regulating such temperatures to a system controller (e.g., system controller 610 shown in 13). The first through third segments 461, 462, 463 may also include respective power rails or buses 494, 495, 496 for receiving power from an external voltage or current source, which may be part of the system controller (e.g., system controller 610). As noted above, the first through third segments 461, 462, 463 are preferably radiolucent (e.g. X-ray transparent or translucent) and designed using primarily carbon fiber materials (e.g., carbon films, carbon nanotubes, etc.).

The heating element 460 according to the fourth exemplary embodiment further reduces the appearance of artifacts on an X-ray by routing the power rails 494, 495, 496 outside the X-ray area (i.e., the central portion of the heating element 460). Additionally, the heating element 460 provides three (3) times the heating power of a single segment heater, due to the presence of three independently controllable heating segments. Those of ordinary skill in the art will realize that this scalability concept may be expanded to increase heating power by increasing the number of segments in the heater. For example, a heater with four (4), five (5) or six (6) segments may be used to increase heating power.

The heating element 460 according to the fourth exemplary embodiment operates similarly to the heating element 360 according to the third exemplary embodiment, in that a user (e.g., health care professional) selects a desired mattress temperature (e.g., 39° C.), and the OCD controls the power supplied to the heating segments 461, 462, 463 and the oxygen supply, so that a mattress reaches a desired temperature in the shortest possible time given conditions. The main difference being that the OCD limits the power supplied to each of the heating elements 461, 462, 463 to 15 VA or less when oxygen is being supplied. However, the presence of three separate heating segments 461, 462, 463 permits a maximum power limit of 45 VA in oxygen rich environments (as opposed to 30 VA in the heating element 360).

The heating element 460 may be controlled by a control system 600 such as is shown in FIG. 13. One difference would be the addition of a third sensor control line for coupling between one of the sensors 491, 492, 493 and the system controller 610. With the heating element 460 according to the fourth exemplary embodiment, the first control line 611 may be coupled to the power rails or buses 494, 495, 496 to enable the controller 610 to supply power to the heating element 630. Similarly, the second control line 612 may be coupled to the backplane of a warming therapy device (e.g., backplane 51 of the warming therapy device 10), which may include one or more oxygen sensors (e.g., oxygen sensors 630) as noted above. The first sensor control line 631 may be coupled to the first sensor 491 of the heating element, the second sensor control line 632 may be coupled to the second sensor 492, and a third sensor control line (not shown) may be coupled to the third sensor 493. The system controller 610 may be used to set a predetermined mattress temperature for monitoring purposes. Such a predetermined temperature may be set (and changed) by a health care professional using, for example, the display and control unit 640.

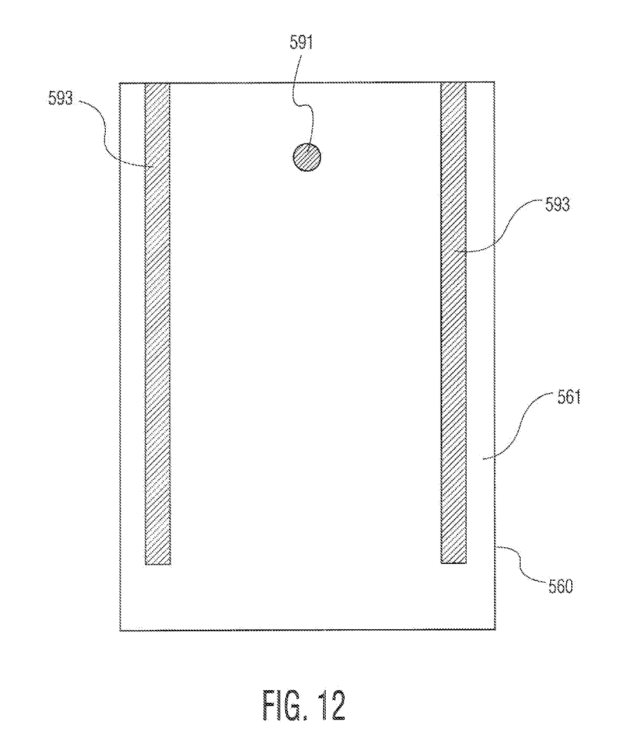

FIG. 12 shows a simplified top cross-section view of a heating element 560 according to a fifth exemplary embodiment of the present invention. The heating element 560 is similar to the heating elements 360 and 460 according to the third and fourth exemplary embodiments in several respects, and in most cases, like reference numerals denote like elements. The heating element 560 may be used in connection with a mattress tray assembly (including a mattress tray and/or mattress) and/or a warming therapy device, such as the mattress tray assemblies 40, 140, and warming therapy devices 10, 100, discussed above with regard to the first and second exemplary embodiments.

Alternatively, from the heating elements 360 and 460, the heating element 560 according to the fifth exemplary embodiment is forted of a single segment 561. However, like the heating elements 360 and 460, the heating element 560 includes a first sensor 591 for sensing the temperature of the segment, and providing a control signal for use in regulating such temperature to a system controller (e.g., system controller 610 shown in FIG. 13). The heating element 560 may also include power rails or buses 593 for receiving power from an external voltage or current source, which may be part of the system controller (e.g., system controller 610). As noted above, the single segment 561 is preferably radiolucent (e.g. X-ray transparent or translucent) and designed using primarily carbon fiber materials (e.g., carbon films, carbon nanotubes, etc.).

The heating element 560 according to the fifth exemplary embodiment operates similarly to the heating elements 360 and 460 described above, in that a user (e.g., health care professional) selects a desired mattress temperature (e.g., 39° C.), and the OCD controls the power supplied to the beating segment 561, and the oxygen Supply, so that a mattress reaches a desired temperature in the shortest possible time given conditions. The main difference being that the OCD limits the power supplied to the single heating element 561 to 15 VA or less when oxygen is being supplied. Thus, the heating element 560 permits a maximum power limit of 15 VA in oxygen rich environments (as opposed to 30 VA in the heating element 360 and 45 VA in the heating element 460).

The heating element 560 may be controlled by a control system 600 such as is shown in FIG. 13. One difference would be that only one of the first and second sensor control lines 631, 632 would be used to connect the system controller 610 to the heating element 630, due to the fact that the heating element includes only one sensor 591. With the heating element 560 according to the fifth exemplary embodiment, the first control line 611 may be coupled to the power rails or buses 593 to enable the controller 610 to supply power to the heating element 630. Similarly, the second control line 612 may be coupled to the back plane of a warming therapy device (e.g., backplane 51 of the warming therapy device 10), which may include one or more oxygen sensors (e.g., oxygen sensors 630) as noted above. The first sensor control line 631 may be coupled to the sensor 591 of the heating element 560. The system controller 610 may be used to set a predetermined mattress temperature for monitoring purposes. Such a predetermined temperature may be set (and changed) by a health care professional using, for example, the display and control unit 640.

Although sensors 391, 392 of the third exemplary embodiment, sensors 491, 492, 493 of the fourth exemplary embodiment, and sensor 591 of the fifth exemplary embodiment are shown in specific locations on the respective heating elements 360, 460, 560, those of ordinary skill will realize that such sensors may be disposed at any suitable location on or near the heaters, so long as accurate temperature readings can be obtained thereby. Further, although the beating elements 360, 460, 560 described above show heating segments of one, two and three portions, those of ordinary skill will realize that the present invention is not so limited, and that heaters with four or more segments are contemplated by the present invention. Finally, although the heating elements 360, 460, 560 according to the third through fifth exemplary embodiments are shown and described above with reference to associated warming therapy devices 10, 110 of specific configurations, those of ordinary skill in the art will realize that the heating elements 360, 460, 560 may be integrated into any suitable mattress, incubator, warmer, medical treatment device or other equivalent apparatus.

Although the invention has been described in terms of exemplary embodiments, it is not limited thereto. Rather, the appended claims should be construed broadly to include other variants and embodiments of the invention which may be made by those skilled in the art without departing from the scope and range of equivalents of the invention. This disclosure is intended to cover any adaptations or variations of the embodiments discussed herein.

What is claimed is:

1. An apparatus comprising:
a patient support assembly; and
at least one heating element coupled to the patient support assembly, said at least one heating element formed of a plurality of heating cells.

2. The apparatus of claim 1, wherein each of the plurality of heating cells comprise at least one heating element and at least one sensor.

3. The apparatus of claim 1, wherein the plurality of heating cells are arranged adjacent one another in a matrix.

4. The apparatus of claim 1, further comprising:
a mattress; and,
a mattress tray for receiving the mattress,
wherein said at least one heating element is disposed within the mattress.

5. The apparatus of claim 4, further comprising an X-ray cassette disposed beneath the mattress tray.

6. The apparatus of claim 1, wherein each of the plurality of heating cells are formed from X-ray transparent material.

7. The apparatus of claim 1, further comprising:
a mattress; and,
a mattress tray for receiving the mattress;
wherein said at least one heating element is disposed between the mattress and the mattress tray.

8. The apparatus of claim 7, further comprising, an X-ray cassette disposed beneath the mattress tray.

9. The apparatus of claim 1, wherein the at least one heating element is formed of a plurality of heating segments.

10. The apparatus of claim 9, wherein the at least one heating element is formed of at least two separate heating segments.

11. The apparatus of claim 9, wherein the at least one heating element is formed of at least three separate heating segments.

12. An apparatus comprising:
a patient support assembly; and,
at least one heating element coupled to the patient support assembly, said at least one heating element formed of an insulating layer and first and second support layers, such that the insulating layer is disposed between the first and second support layers.

13. The apparatus of claim 12, wherein the at least one heating element includes at least two conductive bus bars disposed on opposing sides of the at least one heating element.

14. The apparatus of claim 12, wherein the at least one heating element further comprises:
a heater layer; and,
a grid layer,
wherein the heater layer is disposed between the first support layer and the insulating layer, and the grid layer is disposed between the second support layer and the insulating layer.

15. The apparatus of claim 14, wherein the grid layer comprises a thermocouple grid layer.

16. The apparatus of claim 14, further comprising a controller coupled to the grid layer, wherein the controller operates to sense the temperature at the grid layer, and control the power applied to the at least one heating element.

17. A heating element comprising:
a first support layer;
a heater layer coupled to the first support layer;
an insulating layer coupled to the heater layer;
a grid layer coupled to the insulating layer; and
a second support layer coupled to the grid layer.

18. The heating element of claim 17, further comprising at least two conductive bus bars disposed on opposing sides of the heating element.

19. An apparatus comprising:
a patient support assembly;
at least one heating element coupled to the patient support assembly, said at least one heating element formed of a plurality of heating segments.

20. The apparatus of claim 19, wherein the at least one heating element is formed of at least two separate heating segments.

21. The apparatus of claim 19, wherein the at least one heating element is formed of at least three separate heating segments.

22. The apparatus of claim 19, wherein each of the plurality of heating segments includes power rails disposed on opposing sides thereof, and at least one temperature sensor coupled thereto.

23. The apparatus of claim 19, further comprising at least one oxygen sensor coupled to the at least one heating element.

24. An apparatus comprising:
a patient support assembly;
at least one heating element coupled to the patient support assembly, said at least one heating element including power rails disposed on opposing sides thereof; and at least one temperature sensor coupled thereto.

25. The apparatus of claim 24, further comprising at least one oxygen sensor coupled to the at least one heating element.

26. A control system comprising:
at least one heating element;
a system controller coupled to the at least one heating element; and,
at least one oxygen sensor coupled to the system controller,
wherein the at least one oxygen sensor and the system controller operate to control the power applied to the at least one heating element.

27. The control system of claim 26, wherein the at least one heating element formed of at least two separate heating segments.

28. The control system of claim 26, wherein the at least one heating element is formed of at least three separate heating segments.

29. An apparatus comprising:
a patient support assembly; and,
at least one heating element coupled to the patient support assembly; and
a control system coupled to the at least one heating element, the control system including a system controller coupled to the at least one heating element, and at least one oxygen sensor coupled to the system controller, wherein the at least one oxygen sensor and the system controller operate to control the power applied to the at least one heating element.

30. A method of providing warming therapy to a patient, the method comprising:
providing at least one heating element in proximity to a patient; and,
selectively activating the at least one heating element, so that the patient is heated to a predetermined level.

31. The method of claim 30, comprising the further steps of:
monitoring the oxygen level around the patient; and,
controlling the heating of the at least one heating element so as to not exceed predetermined voltage level at which the oxygen would be subject to ignition.

32. The method of claim 30, wherein the at least one beating element comprises a plurality of heating cells, and wherein the step of selectively activating the at least one heating element comprises selectively activating individuals ones of the plurality of heating cells.

33. The method of claim 30, wherein the at least one heating element is formed of a plurality of separate heating segments, and wherein the step of selectively activating the at least one heating element comprises selectively activating individuals ones of the plurality of heating segments.
